(19) United States
(12) Patent Application Publication
(10) Pub. No.: US 2014/0378875 A1
(43) Pub. Date: Dec. 25, 2014

(54) APPARATUS AND METHOD FOR SELECTIVE HEATING OF A TISSUE

(71) Applicant: VENUS CONCEPT LTD., Karmiel (IL)

(72) Inventors: Oded Ron Edoute, Tel Aviv (IL); Orit Ron Edoute, Tel Aviv (IL)

(21) Appl. No.: 14/342,571

(22) PCT Filed: Sep. 5, 2012
(86) PCT No.: PCT/IL2012/000328

§ 371 Date: Sep. 4, 2014

Related U.S. Application Data
(60) Provisional application No. 61/531,026, filed on Sep. 5, 2011.

(51) Int. Cl. A61N 2/00; A61N 5/00; A61N 2/02; A61N 7/02

(52) U.S. Cl. CPC A61N 2/004; A61N 7/02; A61N 5/00; A61N 2/02
USPC 601/3; 607/103; 607/101

(57) ABSTRACT

An apparatus for selective heating of a biological tissue comprising a plurality of gapped toroidal solenoids having a common gap area to provide a sum of magnetic fields created by the solenoids in common localization. Each solenoid comprises a toroidally configured magnetic core and an electric coil thereupon. The coil is connectable to a generator of electric pulses. The solenoid plurality is accommodated within a housing having an opening configured for emitting solenoid electromagnetic radiation.

### FIELD OF THE INVENTION

The present invention relates to methods of heating a biological tissue and, more specifically, to electromagnetic methods of local tissue heating.

### BACKGROUND OF THE INVENTION

The present invention provides means and method for providing energy (e.g., heat) to a predetermined area/surface/volume of a desired tissue.

### SUMMARY OF THE INVENTION

It is hence one object of the invention to disclose an apparatus for selective heating of a biological tissue. The aforesaid apparatus comprises (a) a plurality of gapped toroidal solenoids having a common gap area to provide a sum of magnetic fields created by said solenoids in common localization; each solenoid comprises a toroidally configured magnetic core and an electric coil thereupon; said coil connectable to a generator of electric pulses; and (b) a housing accommodating said solenoid plurality having an opening configured for emitting solenoid electromagnetic radiation.

Another object of the invention is to disclose the plurality of gapped toroidal solenoids comprising at least two sets of said solenoids having common gap area spaced apart relative to each other.

A further object of the invention is to disclose a shape of said opening selected from a group consisting of circular, oval, rectangular, triangular, polygonal having n ribs where n is greater than 3 and any combination thereof.

A further object of the invention is to disclose the apparatus provided with vacuuming means for air suction of a patient's body to said apparatus.

A further object of the invention is to disclose an apparatus for selective heating of a biological tissue. The aforesaid apparatus comprises (a) a plurality of gapped toroidal solenoids having a common gap area to provide a sum of magnetic fields created by said solenoids in common localization; each solenoid comprises a toroidally configured hollow member and an electric coil accommodated within said hollow member; said coil connectable to a generator of electric pulses; and (b) a housing accommodating said solenoid plurality having an opening configured for emitting solenoid electromagnetic radiation.

A further object of the invention is to disclose at least one toroidal solenoid provided with an ultrasound transducer connectable to an ultrasound generator.

A further object of the invention is to disclose at least one toroidal solenoid provided with an RF transducer connectable to an RF generator.

A further object of the invention is to disclose method of selective heating of a biological tissue. The aforesaid method comprising the steps of: (a) providing an apparatus comprising (i) a plurality of gapped toroidal solenoids having a common gap area to provide a sum of magnetic fields created by said solenoids in common localization; each solenoid comprises a toroidally configured magnetic core and an electric coil thereupon; said coil connectable to a generator of electric pulses; and (ii) a housing accommodating said solenoid plurality having an opening configured for emitting solenoid electromagnetic radiation; (b) attaching said apparatus opening to said tissue to be heated; (c) heating a biological tissue by electromagnetic radiation generated by said toroidal solenoids.

A further object of the invention is to disclose the method comprising a step of vacuuming resulting in air suction of a patient's body to said apparatus.

A further object of the invention is to disclose a method of selective heating of a biological tissue; said method comprising the steps of: (a) providing an apparatus comprising (i) a plurality of gapped toroidal solenoids having a common gap area to provide a sum of magnetic fields created by said solenoids in common localization; each solenoid comprises a toroidally configured hollow member and an electric coil accommodated within said hollow member; said coil connectable to a generator of electric pulses; and (ii) a housing accommodating said solenoid plurality having an opening configured for emitting solenoid electromagnetic radiation; (b) attaching said apparatus opening to said tissue to be heated; (c) heating a biological tissue by electromagnetic radiation generated by said toroidal solenoids.

A further object of the invention is to disclose the method comprising a step of transmitting ultrasound waves.

A further object of the invention is to disclose method comprising a step of transmitting RF waves.

The present invention provides an electronic device which comprise of hardware and accompanied software and energy sources that can emit energy fields focused into a region located in proximity to the said device.

The purpose of the invention is to create a well defined and bounded region on the surface of the skin (or alternatively within a volume of tissue) which will be radiated (or emitted) with a relatively strong energy fields sourced from emitters located around it and emits energy fields that entirely or partly appears in said bounded region.

The local high level energy fields are achieved by the summation of overlapped fields.

It is to be understood that the phenomena of summation of overlapped fields as defined by the present invention refers to the application of different types of energy (from different sources) onto said bounded region. Eventually, said bounded region is radiated with a function G which is a function of the applied energies.

According to one embodiment, function G is a linear combination of the different applied energies.

According to one embodiment, function G is the sum of the different applied energies.

It is important to provide the energy emitters in a predetermined configuration (in terms of locations and orientation) such that, outside said bounded region, there will be less overlapped energy fields and therefore less heating. Such a feature is of high importance in order to minimize (and even eliminates) any damage to the surrounding tissue.

One of the main objects of the present invention is to avoid unnecessary thermal damage to the surrounding tissues (outside of the desired region to be treated).

This invention is useful in tissue treatment since, it provides a technique to induce a local strong energy field (e.g., heating) in a portion of a tissue with minimal (or even without) affecting its surroundings. It can be used for example to ablate a tumor without damaging its surrounding tissue.

### BRIEF DESCRIPTION OF THE INVENTION

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

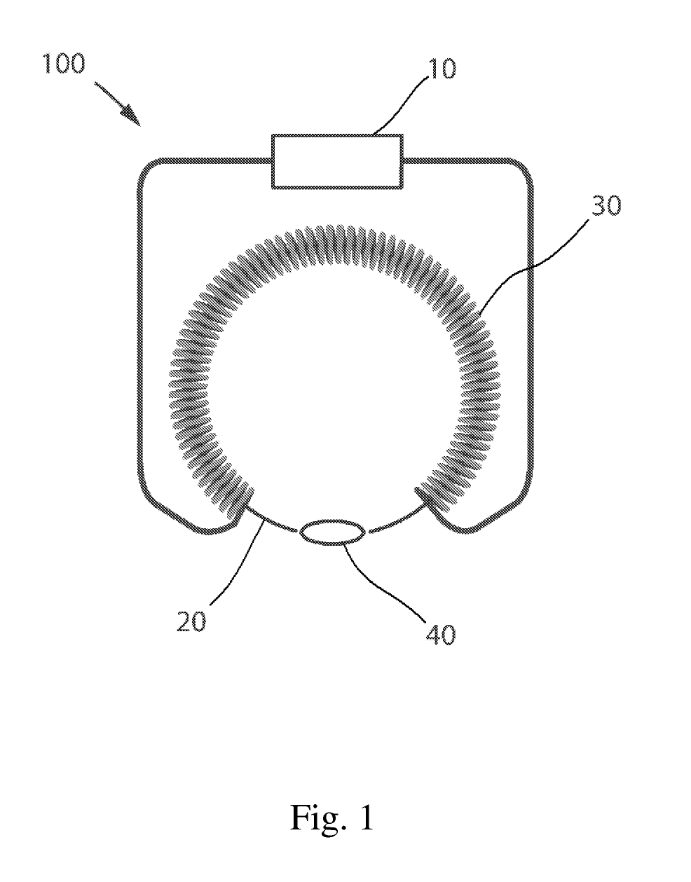

FIG. 1 illustrates a single electro-magnet coil.

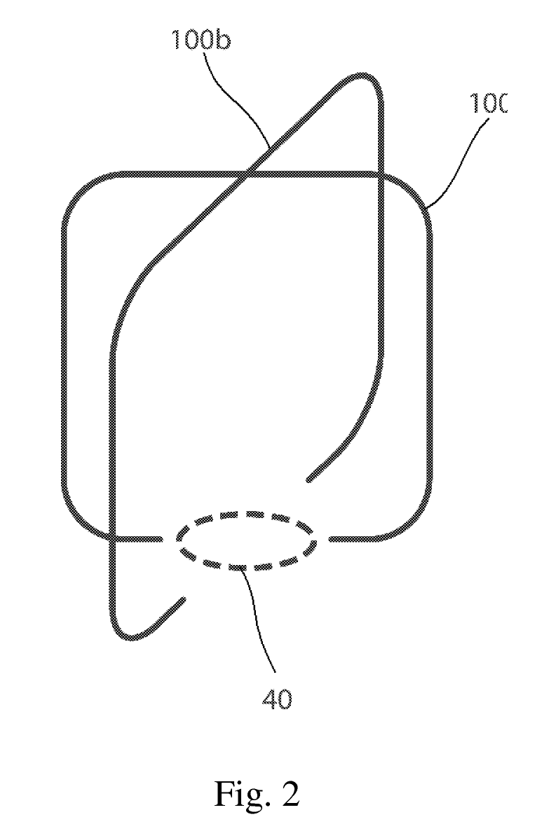

FIG. 2 illustrates two gapped toroidal wound electromagnets.

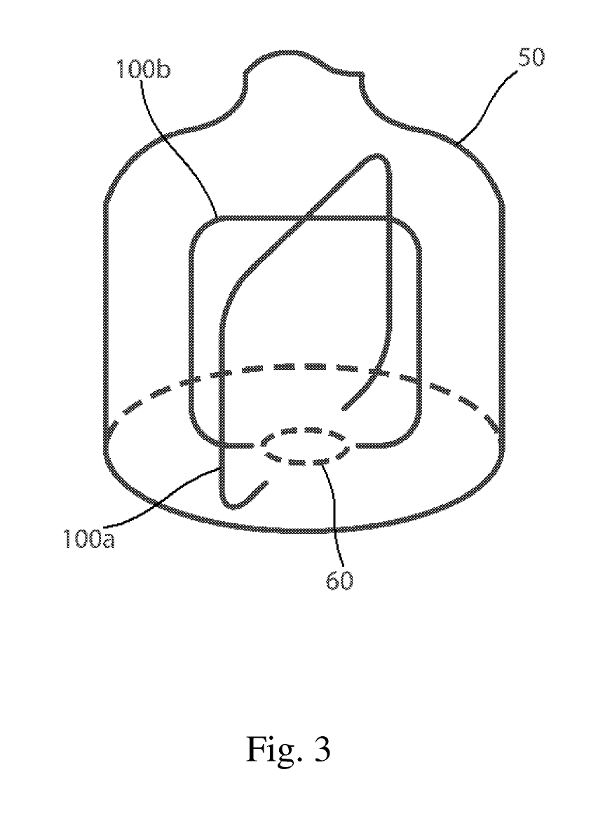

FIG. 3 illustrates the enclosing of the electromagnets together into a hand piece.

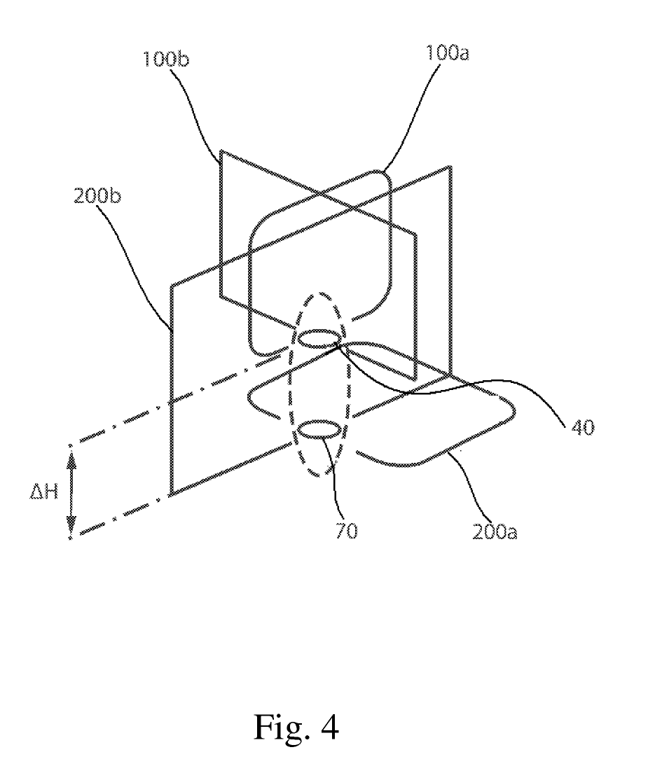

FIG. 4 illustrates are two sets of solenoids in two different heights.

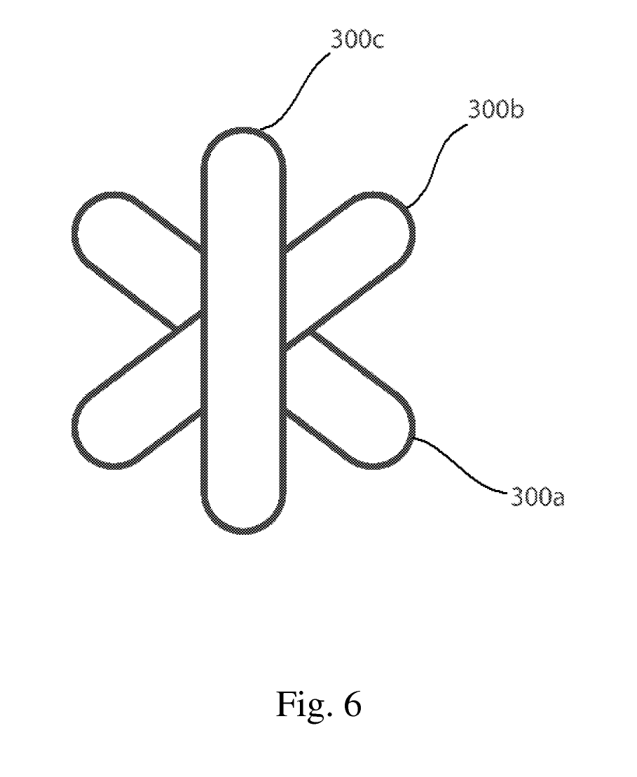

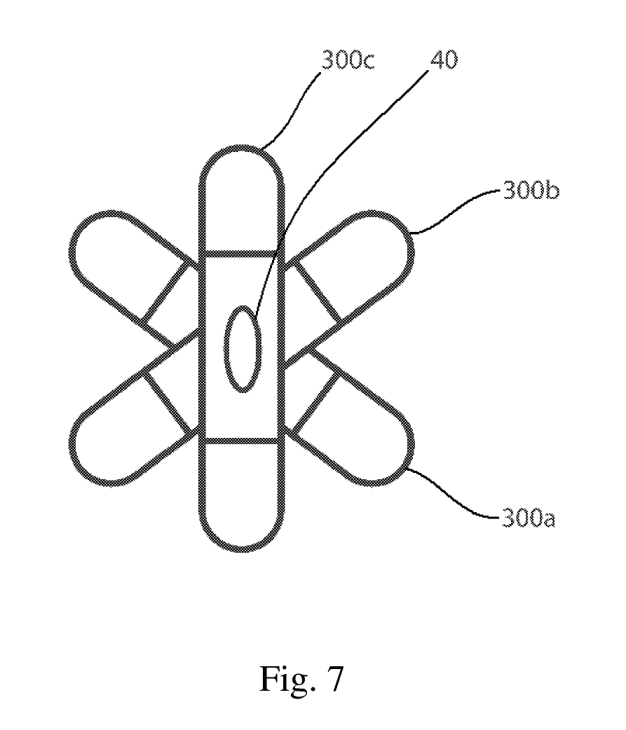

FIGS. 6 and 7 illustrate another embodiment of the present invention in which a combination of 3 types of directional energy.

### DETAIL DESCRIPTION OF THE INVENTION

The purpose of the invention is to create a well defined and bounded region on the surface of the skin (or alternatively within a volume of tissue) which will be radiated (or emitted) with a relatively strong energy fields sourced from emitters located around it and emits energy fields that entirely or partly appears in said bounded region.

The local high level energy fields are achieved by the summation of overlapped fields.

It is to be understood that the phenomena of summation of overlapped fields as it is to be understood that the phenomena of summation of overlapped fields as defined by the present invention refers to the application of different types of energy (from different sources) onto said bounded region. Said bounded region is irradiated with a function G which is a function of the applied energies.

According to one embodiment, function G is a linear combination of the different applied energies.

According to one embodiment, function G is the sum of the different applied energies.

It is important to provide the energy emitters in a predetermined configuration (in terms of locations and orientation) such that, outside said bounded region, there will be less overlapped energy fields and therefore less heating. Such a feature is of high importance in order to minimize (and even eliminates) any damage to the surrounding tissue. One of the main objects of the present invention is to avoid unnecessary thermal damage to the surrounding tissues (outside of the desired region to be treated).

This invention is useful in tissue treatment since it provides a technique to induce a local strong energy field (e.g., heating) in a portion of a tissue without affecting its surroundings. It can be used for example to ablate a tumor without damaging its surrounding tissue.

The types of energy fields to be used can be one or combination of: magnetic, electric, mechanical, radiation, etc.

The shape of the said bounded region depends on the location, orientation, types and physical properties of the energy emitters, mode of operation and environmental conditions.

The following provides examples as for the predetermined arrangement of the energy emitters.

### EXAMPLE 1

A first example of a 3D arrangement of a group of gapped toroidal solenoids mounted so that their gaps enclose a bound region of an imaginary volume. Said volume is shaped as a cylinder combined with two ultrasound transducers mounted on the two bases of said cylinder facing each other. Simultaneous activation of the above set up will produce a bounded region of overlapped fields of energy which induce relatively stronger heating compared to induced heating in rest of the surrounding space.

### EXAMPLE 2

In case that the device is positioned close to a living tissue and is operated in a way that said bounded region is positioned inside a living tissue, and the energy emitters are of the type of gapped toroidal solenoids that emit magnetic pulses, which will induce electrical currents in the intercellular plasma (which is comprised of water, ions, polarized molecules and free electrons).

These currents will produce local heating caused by the collisions among the said moving particles and the surroundings. In other words the device produces local tissue heating none invasively.

The device, when operated using low amplitude of the magnetic pulses, may be used to examine Motor Evoked Potentials (MEP) of superficial muscles (i.e. Muscles of expression).

According to another embodiment, the device is used for cosmetic purposes.

at present, the gold standard for tightening a client's face (namely the face Muscles of expression) is by injecting said client with Botox (which paralyzes the muscles chemically)

The present invention can address such treatment and achieve similar results, using the device and by heating the muscles of expression's motor nerves.

The above alternative is based on the two capabilities of the device:
1) Stimulating the motor nerve of the muscle, by magnetic induction of limited amount of electric current. Said electric current triggers the nerve and cause contraction of the muscle. The operator may identify (e.g. visually) that the target muscle was triggered and device's focal region is located properly for further possible treatment of the tissue bounded in the said focal region. The magnetic pulse may be calibrated to yield the mention current.
2) Heating of the above mentioned tissue. The same is performed by emitting e.g., a train of magnetic pulses, ultrasound, acoustic, RF, laser, light or any combination thereof, which induce high amount of eddie currents in the tissue around the said target point. These currents heat the cells and induce thermal damage to the same. Said damage is either permanently or temporarily for certain period until natural recovery spontaneity may be achieved. To verify that the treatment is accomplished, the operator may repeat the above mentioned stimulating technique to demonstrate that the muscle does not contract anymore.

As discussed above, one of the main features of the present invention is to avoid unnecessary damage to other tissue parts which are not involved in the muscle contraction phenomena, it is recommended to use a technique of dividing the emitted magnetic field into a few weaker fields which are overlapped and summed up only in the focal region.

This choice ensures that the maximum heating occurs only in the overlapped region and the rest of tissue is subjected to partial heating.

It should be understood that the focal region can on the surface of the skin but also in different depths of the same.

The following examples are provided so as to better understand the invention.

Reference is now made to FIG. 1 which illustrates a single electro-magnet coil. The coil 30 residing on a core 20 is coupled to a pulse generator 10 so as to enable to provision of electromagnetic pulses.

According to one embodiment of the present invention, a gap 40 is provided between the two ends of the core 20. Said gap 40 is to be radiated with energy (e.g., heat).

Reference is now made to FIG. 2, which illustrates a second embodiment of the present invention. To paralyze face Muscles of expression, the device comprises two gapped toroidal wound electromagnets 100a and 100b (=solenoids) in which the coils wires are coated with electrical isolation and are designed to conduct alternate current of suitable electrical characteristic. The core material is optimized to the pulses frequency.

According to one embodiment of the present invention one of the toroid's inner diameter is smaller than the other.

According to a specific embodiment, the inner diameter of one toroid is about 6 centimeters and the other's is 4 centimeters. The two cores width is about 1 centimeter and the gaps width is about 1 centimeter. However, it should be understood that the dimension of the toroid and the cores are variable.

Again, as in FIG. 2, the two electro-magnets are position and oriented in a configuration in which the two cross each other; and a 3D gap informed therebetween.

Said gap is to be radiated with energy and heated.

Reference is now made to FIG. 3 which illustrates the enclosing of the two electromagnets 100a and 100b together into a hand piece 52 in a way that their gaps overlapped and a cubical opening space 60 of 1 centimeter width is formed.

According to another embodiment, the shape of the opening 60 can be selected from a group consisting of circular, oval, rectangular, triangular, polygonal having n ribs where n is greater than 3 and any combination thereof.

In this opening space, the maximum temperature will be created.

According to a further embodiment, the hand piece 50 is designed in a way that the gaps are aligned with its outer surface and forms rectangular opening 60 on its surface.

According to another embodiment, the gaps are not aligned with its outer surface and a volume is created.

In addition the hand piece may contain an internal vacuum generating mechanism that when the hand piece is in contact with the patient's face skin, the skin is elevated and a portion of the tissue is placed inside the said gaps volume.

It should be pointed and emphasized that according to one embodiment of the present invention, the hand piece may, instead or in addition to said vacuum generating mechanism, any mechanical means adapted to ‘pinch’ the tissue so as to provide a volume of treatment.

Such mechanical means can be selected from e.g., forceps, pliers, pincers and any combination thereof.

The electromagnets are e.g., energized by electric current pulses provided from external generator;

In order to locate the relevant point in the tissue, the operator will attach the hand piece to several points onto the skin and will stimulate them in a low intensity pulses until he identifies visually muscle contraction, then raise the pulses intensity to higher level and send a train of strong magnetic pulses into the skin for a limited period. This treatment may result with heating of the target muscle.

In order to build a bigger shaped region of treatment, a combination of solenoids can be mounted inside the hand piece in a way that their gaps locations will create a continuous volume in the bounded space.

Reference is now made to FIG. 4 which illustrates the same. In the figure, there are two sets of solenoids (100a, 100b) and (200a 200b) in two different heights (ΔH). While all solenoids will emit magnetic pulses, the overlapped treatment regions will be shaped as a cylinder or cigar.

According to another embodiment of the present invention, the hand piece will enclosed a combined energy sources into one element (=probe).

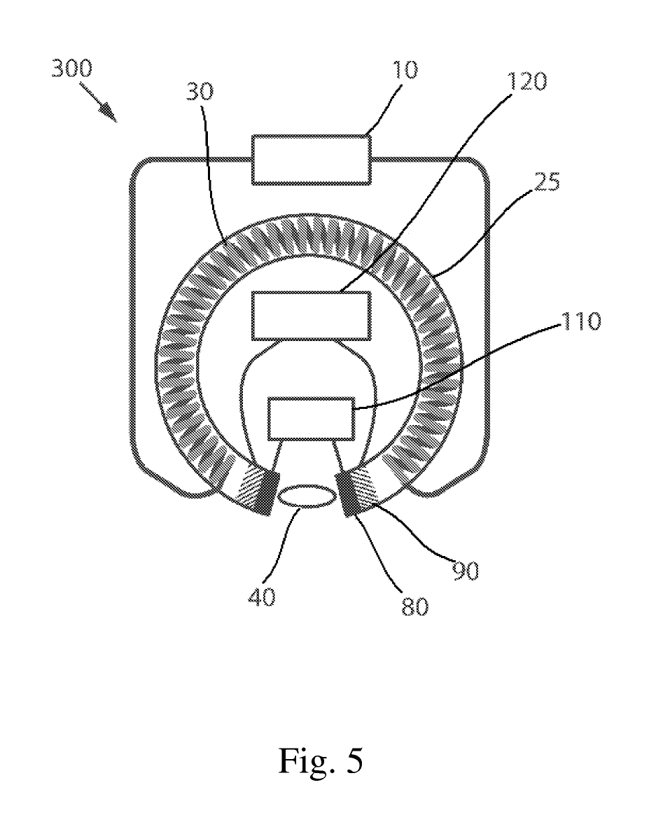

Reference is now made to FIG. 5, presenting a hollow gapped toroid shaped pipe 25 which contains an internal coil 30 of isolated wires. The aforesaid pipe terminates (on the two gap faces) with a couple of piezo electric ultra sound transducers housed inside a couple of metallic cases 90. The transducers are connected to an ultrasound generator 120. Additionally, electrodes 90 connected to an RF signal generator provide electromagnetic energy to the gap 40.

Thus, the device 300 emits 3 types of directional energy, specifically: magnetic, mechanical and electrical energy.

A combination of 3 elements organized in a way that their gaps are overlapped is illustrated in FIG. 5. Such a combination provides region of treatment in which the heating is stronger than around it.

Reference is now made to FIGS. 6 and 7, presenting top and bottom views of an exemplary arrangement of three devices 300, respectively. The aforesaid arrangement can be used for cosmetic treatment of patient in a topical manner.

In accordance with one embodiment of the present invention, an apparatus for selective heating of a biological tissue is disclosed. The aforesaid apparatus comprising (a) a plurality of gapped toroidal solenoids having a common gap area to provide a sum of magnetic fields created by said solenoids in common localization; each solenoid comprises a toroidally configured magnetic core and an electric coil thereupon; said coil connectable to a generator of electric pulses; and (b) a housing accommodating said solenoid plurality having an opening configured for emitting solenoid electromagnetic radiation.

In accordance with another embodiment of the present invention, the plurality of gapped toroidal solenoids comprises at least two sets of said solenoids having common gap area spaced apart relative to each other.

In accordance with a further embodiment of the present invention, a shape of said opening is selected from a group consisting of circular, oval, rectangular, triangular, polygonal having n ribs where n is greater than 3 and any combination thereof.

In accordance with a further embodiment of the present invention, apparatus is provided with vacuuming means for air suction of a patient's body to said apparatus.

In accordance with a further embodiment of the present invention, an apparatus for selective heating of a biological tissue is disclosed. The aforesaid apparatus comprises (a) a plurality of gapped toroidal solenoids having a common gap area to provide a sum of magnetic fields created by said solenoids in common localization; each solenoid comprises a toroidally configured hollow member and an electric coil accommodated within said hollow member; said coil connectable to a generator of electric pulses; and (b) a housing accommodating said solenoid plurality having an opening configured for emitting solenoid electromagnetic radiation.

In accordance with a further embodiment of the present invention, at least one toroidal solenoid is provided with an ultrasound transducer connectable to an ultrasound generator.

In accordance with a further embodiment of the present invention, at least one toroidal solenoid is provided with an RF transducer connectable to an RF generator.

In accordance with a further embodiment of the present invention, a method of selective heating of a biological tissue is disclosed. The aforesaid method comprising the steps of: (a) providing an apparatus comprising (i) a plurality of gapped toroidal solenoids having a common gap area to provide a sum of magnetic fields created by said solenoids in common localization; each solenoid comprises a toroidally configured magnetic core and an electric coil thereupon; said coil connectable to a generator of electric pulses; and (ii) a housing accommodating said solenoid plurality having an opening configured for emitting solenoid electromagnetic radiation; (b) attaching said apparatus opening to said tissue to be heated; (c) heating a biological tissue by electromagnetic radiation generated by said toroidal solenoids.

In accordance with a further embodiment of the present invention, the method comprises a step of vacuuming resulting in air suction of a patient's body to said apparatus.

In accordance with a further embodiment of the present invention, a method of selective heating of a biological tissue is disclosed. The aforesaid method comprises the steps of: (a) providing an apparatus comprising (i) a plurality of gapped toroidal solenoids having a common gap area to provide a sum of magnetic fields created by said solenoids in common localization; each solenoid comprises a toroidally configured hollow member and an electric coil accommodated within said hollow member; said coil connectable to a generator of electric pulses; and (ii) a housing accommodating said solenoid plurality having an opening configured for emitting solenoid electromagnetic radiation; (b) attaching said apparatus opening to said tissue to be heated; and (c) heating a biological tissue by electromagnetic radiation generated by said toroidal solenoids.

In accordance with a further embodiment of the present invention, the method comprises a step of transmitting ultrasound waves.

In accordance with a further embodiment of the present invention, the method comprises a step of transmitting RF waves.

1. An apparatus for selective heating of a biological tissue, said apparatus comprising
a. a plurality of gapped toroidal solenoids having a common gap area to provide a sum of magnetic fields created by said solenoids in common localization; each solenoid comprises a toroidally configured magnetic core and an electric coil thereupon; said coil connectable to a generator of electric pulses; and
b. a housing accommodating said solenoid plurality having an opening configured for emitting solenoid electromagnetic radiation.

2. The apparatus according to claim 1, wherein said plurality of gapped toroidal solenoids comprises at least two sets of said solenoids having common gap area spaced apart relative to each other.

3. The apparatus according to claim 1, wherein a shape of said opening is selected from a group consisting of circular, oval, rectangular, triangular, polygonal having n ribs where n is greater than 3 and any combination thereof.

4. The apparatus according to claim 1 provided with vacuuming means for air suction of a patient's body to said apparatus.

5. An apparatus for selective heating of a biological tissue; said apparatus comprising
a. a plurality of gapped toroidal solenoids having a common gap area to provide a sum of magnetic fields created by said solenoids in common localization; each solenoid comprises a toroidally configured hollow member and an electric coil accommodated within said hollow member; said coil connectable to a generator of electric pulses; and
b. a housing accommodating said solenoid plurality having an opening configured for emitting solenoid electromagnetic radiation.

6. The apparatus according to claim 5, wherein said plurality of gapped toroidal solenoids comprises at least two sets of said solenoids having common gap area spaced apart relative to each other.

7. The apparatus according to claim 5, wherein a shape of said opening is selected from a group consisting of circular, oval, rectangular, triangular, polygonal having n ribs where n is greater than 3 and any combination thereof.

8. The apparatus according to claim 5 provided with vacuuming means for air suction of a patient's body said apparatus.

9. The apparatus according to claim 5, wherein at least one toroidal solenoid is provided with an ultrasound transducer connectable to an ultrasound generator.

10. The apparatus according to claim 5, wherein at least one toroidal solenoid is provided with an RF transducer connectable to an RF generator.

11. A method of selective heating of a biological tissue; said method comprising the steps of:
a. providing an apparatus comprising
i. a plurality of gapped toroidal solenoids having a common gap area to provide a sum of magnetic fields created by said solenoids in common localization; each solenoid comprises a toroidally configured magnetic core and an electric coil thereupon; said coil connectable to a generator of electric pulses; and
ii. a housing accommodating said solenoid plurality having an opening configured for emitting solenoid electromagnetic radiation;
b. attaching said apparatus opening to said tissue to be heated;
c. heating a biological tissue by electromagnetic radiation generated by said toroidal solenoids.

12. The method according to claim 11 comprising a step of vacuuming resulting in air suction of a patient's body to said apparatus.

13. A method of selective heating of a biological tissue; said method comprising the steps of:
a. providing an apparatus comprising
i. a plurality of gapped toroidal solenoids having a common gap area to provide a sum of magnetic fields created by said solenoids in common localization; each solenoid comprises a toroidally configured hollow member and an electric coil accommodated within said hollow member; said coil connectable to a generator of electric pulses; and
ii. a housing accommodating said solenoid plurality having an opening configured for emitting solenoid electromagnetic radiation;
b. attaching said apparatus opening to said tissue to be heated;
c. heating a biological tissue by electromagnetic radiation generated by said toroidal solenoids.

14. The method according to claim 13 comprising a step of transmitting ultrasound waves.

15. The method according to claim 13 comprising a step of transmitting RF waves.
